(19) United States
(12) Patent Application Publication
(10) Pub. No.: US 2022/0382394 A1
(43) Pub. Date: Dec. 1, 2022

(54) TRACKBALL APPARATUS, MEDICAL INPUT APPARATUS, AND ULTRASONIC IMAGING APPARATUS

(71) Applicant: GE Precision Healthcare LLC, Wauwatosa, WI (US)

(72) Inventors: Xiaoren Wang, Wuxi (CN); Yue Wu, Wuxi (CN); Junping Zou, Wuxi (CN); Zhiyuan Peng, Wuxi (CN)

(21) Appl. No.: 17/751,326

(22) Filed: May 23, 2022

(30) Foreign Application Priority Data
May 31, 2021 (CN) 202110602151.4

(51) Int. Cl. G06F 3/0354; A61B 8/00

(52) U.S. Cl. CPC G06F 3/03549; A61B 8/467

(57) ABSTRACT

Embodiments of the present application provide a trackball apparatus, a medical input apparatus, and an ultrasonic imaging apparatus. The trackball apparatus includes a cover plate, a housing and a ball. The cover plate has a first surface and a second surface, where an opening portion passing through the first surface and the second surface is provided at the center of the cover plate, and one or more engaging portions are provided on the second surface. The housing has an accommodating portion, where when the cover plate is mounted on the housing, the one or more engaging portions are located on an outer side of the housing. The ball is accommodated in the accommodating portion in a freely rotatable state, where a part of the ball is exposed from the opening portion. In this way, the trackball apparatus can be easily mounted and detached, thereby facilitating maintenance and product replacement.

### CROSS-REFERENCE TO RELATED APPLICATION SECTION

This application claims priority to Chinese patent application number 202110602151.4, filed on May 31, 2021, the entirety of which is incorporated herein by reference.

### TECHNICAL FIELD

Embodiments of the present application relate to the technical field of medical equipment, and relate in particular to a trackball apparatus, a medical input apparatus, and an ultrasonic imaging apparatus.

### BACKGROUND

In medical equipment (such as an ultrasonic imaging apparatus), an input apparatus is needed, and a trackball is a common input apparatus. The rotation direction and/or speed of a ball rotated by a user can be detected by a pair of encoders corresponding to an X axis and a Y axis that are orthogonally configured. A corresponding cursor or pointer can be displayed on a display screen according to the detected rotation direction and/or speed.

However, the inventor finds that a current trackball apparatus is fixed on an operation panel, and if a circuit such as that of an encoder is damaged, the entire operation panel needs to be detached. Such processing takes a long time and is not easy to perform, so that maintenance and product replacement cannot be performed easily.

To resolve at least one of the above technical problems, embodiments of the present application provide a trackball apparatus, a medical input apparatus, and an ultrasonic imaging apparatus. The trackball apparatus can be easily detached and mounted, so as to facilitate maintenance and product replacement.

### SUMMARY

According to an embodiment, a trackball apparatus includes a cover plate having a first surface and a second surface, wherein an opening portion passing through the first surface and the second surface is provided at the center of the cover plate, and one or more engaging portions are provided on the second surface. The trackball apparatus includes a housing having an accommodating portion, wherein the cover plate contacts an end of the housing via the second surface such that the cover plate is mounted on the housing, and when the cover plate is mounted on the housing, the one or more engaging portions are located on an outer side of the housing. The trackball apparatus includes a ball accommodated in the accommodating portion in a freely rotatable state, wherein a part of the ball is exposed from the opening portion.

According to an embodiment, a medical input apparatus includes a trackball apparatus and an operation panel. The trackball apparatus includes a cover plate having a first surface and a second surface, wherein an opening portion passing through the first surface and the second surface is provided at the center of the cover plate, and one or more engaging portions are provided on the second surface. The trackball apparatus includes a housing having an accommodating portion, wherein the cover plate contacts an end of the housing via the second surface such that the cover plate is mounted on the housing, and when the cover plate is mounted on the housing, the one or more engaging portions are located on an outer side of the housing. The trackball apparatus includes a ball accommodated in the accommodating portion in a freely rotatable state, wherein a part of the ball is exposed from the opening portion. The operation panel has a frame for the trackball apparatus to be mounted therein, wherein one or more position-limiting portions matching the engaging portions of the trackball apparatus are provided inside the frame, and when the trackball apparatus is mounted in the frame, the position-limiting portions are engaged with the engaging portions of the trackball apparatus.

According to an embodiment, an ultrasonic imaging apparatus includes a trackball apparatus and an operation panel. The trackball apparatus includes a cover plate having a first surface and a second surface, wherein an opening portion passing through the first surface and the second surface is provided at the center of the cover plate, and one or more engaging portions are provided on the second surface. The trackball apparatus includes a housing having an accommodating portion, wherein the cover plate contacts an end of the housing via the second surface such that the cover plate is mounted on the housing, and when the cover plate is mounted on the housing, the one or more engaging portions are located on an outer side of the housing. The trackball apparatus includes a ball accommodated in the accommodating portion in a freely rotatable state, wherein a part of the ball is exposed from the opening portion. The operation panel has a frame for the trackball apparatus to be mounted therein, wherein one or more position-limiting portions matching the engaging portions of the trackball apparatus are provided inside the frame, and when the trackball apparatus is mounted in the frame, the position-limiting portions are engaged with the engaging portions of the trackball apparatus.

With reference to the following description and accompanying drawings, specific implementations of the embodiments of the present application are disclosed in detail, and manners in which the principle of the embodiments of the present application is employed are illustrated. It should be understood that the implementations of the present application are not thereby limited in scope. Within the spirit and scope of the appended claims, the implementations of the present application comprise various changes, modifications, and equivalents.

### BRIEF DESCRIPTION OF THE DRAWINGS

The accompanying drawings are included to provide further understanding of embodiments of the present application, constitute a part of the specification, and are used to illustrate implementations of the present application and set forth the principles of the present application together with textual description. Obviously, the accompanying drawings in the following description are merely some embodiments of the present application, and a person of ordinary skill in the art could obtain other implementations according to the accompanying drawings without the exercise of inventive effort. In the accompanying drawings:

FIG. 1 is a schematic diagram of a trackball apparatus according to an embodiment of the present application;

FIG. 2 is another schematic diagram of the trackball apparatus according to an embodiment of the present application;

FIG. 3 is a schematic diagram of a cover plate according to an embodiment of the present application;

FIG. 4 is another schematic diagram of the cover plate according to an embodiment of the present application;

FIG. 5 is another schematic diagram of the cover plate according to an embodiment of the present application;

FIG. 6 is a schematic diagram of a medical input apparatus according to an embodiment of the present application;

FIG. 7 is another schematic diagram of the medical input apparatus according to an embodiment of the present application;

FIG. 8 is a schematic diagram of an operation panel according to an embodiment of the present application;

FIG. 9 is another schematic diagram of the operation panel according to an embodiment of the present application;

FIG. 10 is another schematic diagram of the operation panel according to an embodiment of the present application; and

FIG. 11 is a schematic diagram of an ultrasonic imaging apparatus according to an embodiment of the present application.

### DETAILED DESCRIPTION

The foregoing and other features of the embodiments of the present application will become apparent from the following description with reference to the accompanying drawings. In the description and the accompanying drawings, specific implementations of the present application are specifically disclosed, and part of the implementations in which the principles of the embodiments of the present application may be employed are indicated. It should be understood that the present application is not limited to the described implementations. On the contrary, the embodiments of the present application include all modifications, variations, and equivalents falling within the scope of the appended claims.

In the embodiments of the present application, the terms “first,” “second,” etc. are used to distinguish different elements, but do not represent a spatial arrangement or temporal order etc. of these elements, and these elements should not be limited by these terms. The term “and/or” includes any one of and all combinations of one or more associated listed terms. The terms “comprise,” “include,” “have,” etc. refer to the presence of described features, elements, components, or assemblies, but do not exclude the presence or addition of one or more other features, elements, components, or assemblies.

In the embodiments of the present application, the singular forms “a,” “the,” etc. include plural forms, and should be broadly construed as “a type of” or “a class of” rather than limited to the meaning of “one.” Furthermore, the term “said” should be construed as including both the singular and plural forms, unless otherwise specified in the context. In addition, the term “according to” should be construed as “at least in part according to . . . ,” and the term “based on” should be construed as “at least in part based on . . . ,” unless otherwise specified in the context.

The features described and/or illustrated for one implementation may be used in one or more other implementations in the same or similar manner, combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

In addition, in the following description of the present application, for the convenience of description, a direction extending along a central axis OO′ of an opening portion or parallel to the central axis is called an “axial direction” (an up-down direction or a vertical direction), a radius direction using the axial direction as the center is called a “radial direction” (a horizontal direction), and a direction around the axial direction is called a “circumferential direction”. However, it should be noted that these are just for the convenience of description, and do not limit an orientation of the trackball apparatus during manufacturing and use.

Embodiments of the present application provide a trackball apparatus.

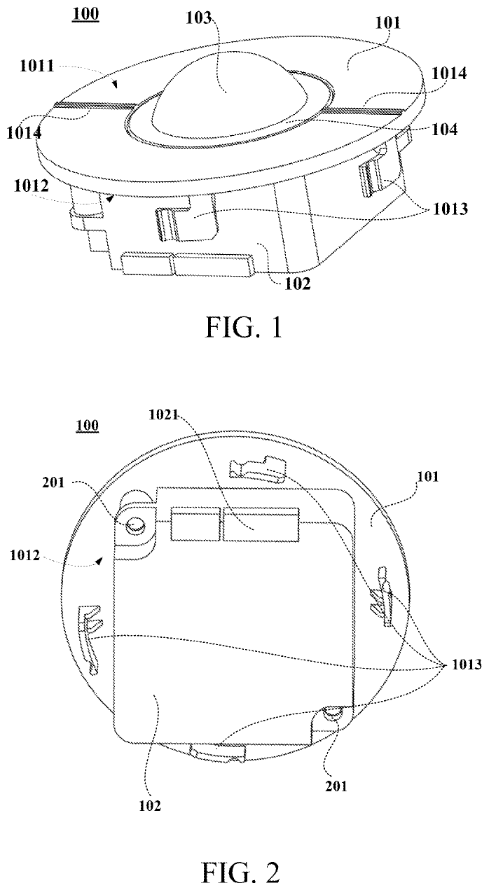

FIG. 1 is a schematic diagram of a trackball apparatus according to an embodiment of the present application, and shows a trackball apparatus 100 viewed from a side; FIG. 2 is another schematic diagram of the trackball apparatus according to an embodiment of the present application, and shows the trackball apparatus 100 viewed from below.

As shown in FIG. 1 and FIG. 2, the trackball apparatus 100 includes:

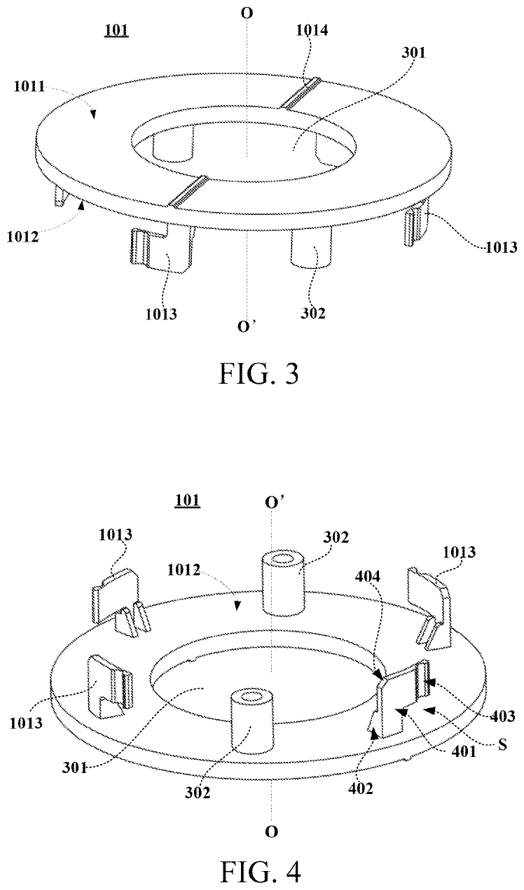

a cover plate 101 having a first surface 1011 and a second surface 1012, wherein an opening portion (please refer to the opening portion 301 in FIG. 3, which is not shown in FIG. 1 because the opening portion is blocked by a ball 103 and a fixing member 104 to be described later) passing through the first surface 1011 and the second surface 1012 is provided at the center of the cover plate 101, and one or more engaging portions 1013 are provided at the second surface 1012;
a housing 102 having an accommodating portion (not shown in FIG. 1 because the accommodating portion is blocked by the cover plate 101), wherein the cover plate 101 contacts an end of the housing 102 via the second surface 1012 such that the cover plate can be mounted on the housing 102, and one or more engaging portions 1013 are located on an outer side of the housing 102 when the cover plate 101 is mounted on the housing 102; and
a ball 103 accommodated in the accommodating portion in a freely rotatable state, wherein a part of the ball 103 is exposed from the opening portion.

In this way, by means of the engaging portions 1013, the trackball apparatus 100 as a whole, including the cover plate 101, the housing 102, and the ball 103, can be mounted on or detached from an operation panel. Therefore, the trackball apparatus can be easily and quickly detached and mounted, thereby facilitating maintenance and product replacement.

It should be noted that FIG. 1 and FIG. 2 merely schematically illustrate the embodiments of the present application, but the present application is not limited thereto. For example, each component may be adjusted appropriately, and some other components may be incorporated or some components may be eliminated. Those skilled in the art could make appropriate variations according to the above content, rather than being limited by the disclosure of FIG. 1 and FIG. 2.

In some embodiments, as shown in FIG. 1, the first surface 1011 of the cover plate 101 has a force receiving portion 1014 for applying a rotational force to the cover plate 101. For example, as shown in FIG. 1, the force receiving portion 1014 is a linear protruding component, but the present application is not limited thereto. For example, the force receiving portion may be a linear recess, a dot-shaped hole portion, or the like.

In this way, the rotational force can be applied to the cover plate, so as to engage or disengage the trackball apparatus conveniently, thereby further facilitating mounting or detachment of the trackball apparatus. In addition, the force receiving portion 1014 can further indicate a mounting state (engaged or disengaged) of the cover plate 101.

In some embodiments, a detector is provided in the accommodating portion of the housing 102, and is used to detect input information generated by rotation of the ball 103. For example, a pair of encoders corresponding to an X axis and a Y axis (X/Y encoder for short hereinafter) may be configured in the accommodating portion. The rotation direction and/or speed of the ball 103 rotated by a user can be detected by the X/Y encoder. A corresponding cursor or pointer can be displayed on a display screen according to the detected rotation direction and/or speed.

As shown in FIG. 2, the housing 102 may further be provided with a socket structure 1021, and the detector may be electrically connected to an external device via the socket structure 1021. For example, power may be obtained from the outside. As another example, detection information may be transmitted to a corresponding device. With respect to the specific structure of the detector, reference can be made to the related art.

In this way, an input function of the trackball apparatus can be implemented, which facilitates mounting or detachment of the trackball apparatus as a whole.

In addition, during daily use by the user, it is also possible to only detach and clean the ball 103.

In some embodiments, as shown in FIG. 1, the trackball apparatus 100 may further include the fixing member 104 having a circular opening (not shown in FIG. 1 as the circular opening is blocked by the ball 103). The diameter of the circular opening of the fixing member 104 is less than that of the ball 103, and the fixing member 104 is detachably connected to the housing 102. When the cover plate 101 is mounted on the housing 102, the fixing member 104 is located inside the opening portion of the cover plate 101, and a part of the ball 103 is exposed from the circular opening of the fixing member 104.

For example, the fixing member is annular in FIG. 1, but the present application is not limited thereto. For example, the fixing member may also be rectangular or square having a circular opening in the center; that is, the fixing member is square outside and circular inside in shape; or a periphery of the fixing member may be irregular, or the like; but the present application is not limited thereto.

In this way, the ball 103 can be removed from the accommodating portion of the housing 102 by detaching the fixing member 104, thereby further facilitating cleaning and maintenance of the inside of the trackball apparatus.

In some embodiments, the cover plate 101 contacts an end of the housing 102 via the mounting portion such that the cover plate can be mounted on the housing 102. For example, as shown in FIG. 2, the housing 102 may have a hole portion 201, and the hole portion 201 corresponds to the mounting portion of the cover plate 101 (shown as a mounting portion 302 in FIG. 3 and FIG. 4). By means of the hole portion 201 and the mounting portion 302, the cover plate 101 can be mounted on the housing 102 through a screw. However, the present application is not limited thereto, and other mounting methods may further be used. For example, the cover plate may further be mounted through an engagement block, a screw thread, or the like, or may be connected by riveting, a hinge, or the like.

In some embodiments, as shown in FIG. 1 and FIG. 2, the cover plate 101 is roughly annular, and the housing 102 is roughly box-shaped. However, the present application is not limited thereto. For example, the cover plate 101 may also be roughly in the shape of a rectangle, and a circular opening portion is provided at the center of the rectangle, while the housing 102 is roughly circular. The shapes of the cover plate and the housing are not limited in the present application. The shapes shown in FIG. 1 and FIG. 2 are used as examples below.

The trackball apparatus 100 as a whole is described above, and the cover plate 101 will be further described below.

FIG. 3 is a schematic diagram of a cover plate according to an embodiment of the present application, and shows the cover plate 101 viewed from an upper side. As shown in FIG. 3, an opening portion 301 is provided at the center of the cover plate 101, a force receiving portion 1014 is provided on the first surface 1011 of the cover plate 101, and one or more engaging portions 1013 are provided on the second surface 1012 of the cover plate 101.

As shown in FIG. 3, one or more mounting portions 302 are further provided on the second surface 1012 of the cover plate 101.

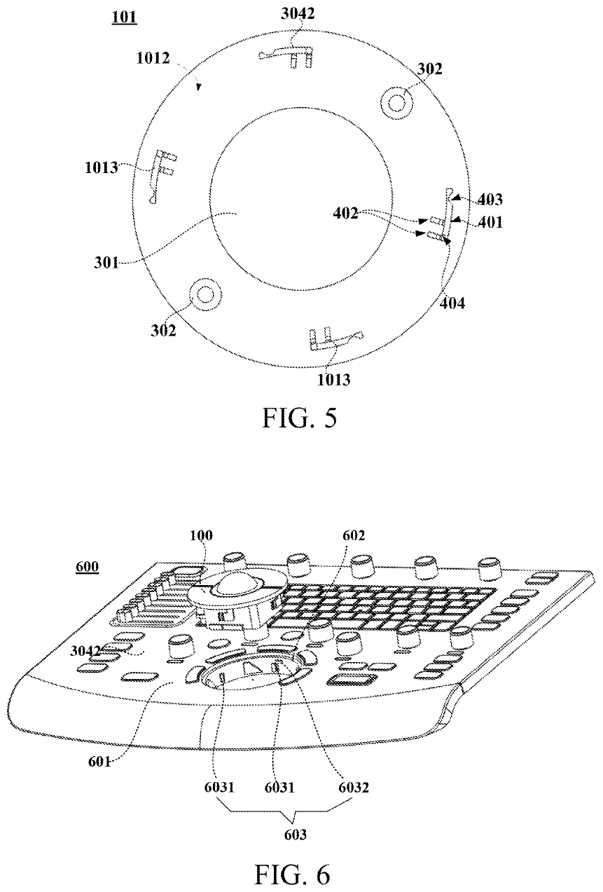

FIG. 4 is another schematic diagram of the cover plate according to an embodiment of the present application, and shows the second surface 1012 of the cover plate 101 viewed from an upper side. FIG. 5 is another schematic diagram of the cover plate according to an embodiment of the present application, and shows the second surface 1012 of the cover plate 101 viewed from above.

As shown in FIG. 4 and FIG. 5, in some embodiments, a plurality of engaging portions 1013 and mounting portions 302 may be provided in a circumferential direction.

For example, as shown in FIG. 4 and FIG. 5, the second surface 302 is provided with two mounting portions 302, which are symmetrically arranged relative to a central axis OO′. The second surface 302 is further provided with four engaging portions 1013, and two of the engaging portions 1013 are symmetrically arranged relative to the central axis OO′, and the other two engaging portions 1013 are also symmetrically arranged relative to the central axis OO′.

In this way, engagement reliability can be further improved, and the trackball apparatus can be more easily mounted or detached.

Only two mounting portions 302 and four engaging portions 1013 are used as an example in FIG. 4 and FIG. 5, but the present application is not limited thereto. For example, more engaging portions may be provided, or only one or more engaging portions may be provided. The number of the engaging portions and the number of the mounting portions are not limited in the present application.

In some embodiments, as shown in FIG. 4 and FIG. 5, the mounting portion 302 is roughly cylindrical, and the engaging portion 1013 is roughly plate-shaped. However, the present application is not limited thereto, and the mounting portion 302 and the engaging portion 1013 may have other shapes. In addition, the engaging portion 1013 may be made of a flexible material, thereby facilitating engagement.

In some embodiments, as shown in FIG. 4 and FIG. 5, one engaging portion 1013 is used as an example. The engaging portion 1013 has a plate-shaped portion 401 extending from the second surface 302 in an axial direction, and a recess 403 extending in the axial direction is provided on a peripheral side of the plate-shaped portion 401. In this way, engagement is performed through the recess 403, so that engagement reliability can be further improved, and the trackball apparatus can be more easily mounted or detached.

In some embodiments, as shown in FIG. 4 and FIG. 5, a reinforcing portion 402 extending in a radial direction is further provided on a side of the plate-shaped portion 401 close the second surface 302, and a cut-out portion 404 is provided on another peripheral side of the plate-shaped portion 401.

For example, as shown in FIG. 4 and FIG. 5, two reinforcing portions 402 may be provided, but the present application is not limited thereto. An engaging portion 1013 having the reinforcing portion 402, the recess 403, and the cut-out portion 404 is used as an example for description above, but the present application is not limited thereto. For example, an engaging portion may only be provided with one or a combination of the reinforcing portion, the recess, and the cut-out portion.

In this way, the strength of the engaging portion can be enhanced, and guidance can be performed through the cut-out portion. Engagement reliability can be further improved, and the trackball apparatus can be more easily mounted or detached.

In some embodiments, the recess 403 does not contact the second surface 302, and the cut-out portion 404 is located away from the second surface 302 in the axial direction. For example, as shown in FIG. 4, the recess 403 is provided on an extension portion of the plate-shaped portion 401 that extends in the circumferential direction. The recess 403 is suspended above the second surface 302, and a gap S is present therebetween.

In this way, the elasticity of the engaging portion can be enhanced, and guidance can be performed through the cut-out portion. In addition, engagement can be performed through the gap between the recess and the second surface (as described in the embodiment of a second aspect). Engagement reliability can be further improved, and the trackball apparatus can be more easily mounted or detached.

It should be noted that FIG. 3 to FIG. 5 merely schematically illustrate the embodiments of the present application, but the present application is not limited thereto. For example, each component may be adjusted appropriately, and some other components may be incorporated or some components may be eliminated. Those skilled in the art could make appropriate variations according to the above content, rather than being limited by the disclosure of FIG. 3 to FIG. 5.

The above embodiments merely provide illustrative description of the embodiments of the present application. However, the present application is not limited thereto, and appropriate variations may be made on the basis of the above embodiments. For example, each of the above embodiments may be used independently, or one or more of the above embodiments may be combined.

As can be seen from the above embodiments, the second surface of the cover plate is provided with the engaging portions, and the engaging portions are located on an outer side of the housing when the cover plate is mounted on the housing. In this way, the trackball apparatus as a whole is engaged through the engaging portions, so that the trackball apparatus can be easily and quickly mounted and detached, thereby facilitating maintenance and product replacement.

An embodiment of the present application further provides a medical input apparatus, including an operation panel and the trackball apparatus as described in the forgoing embodiments, and the same content as that of the forgoing embodiments is not repeated herein.

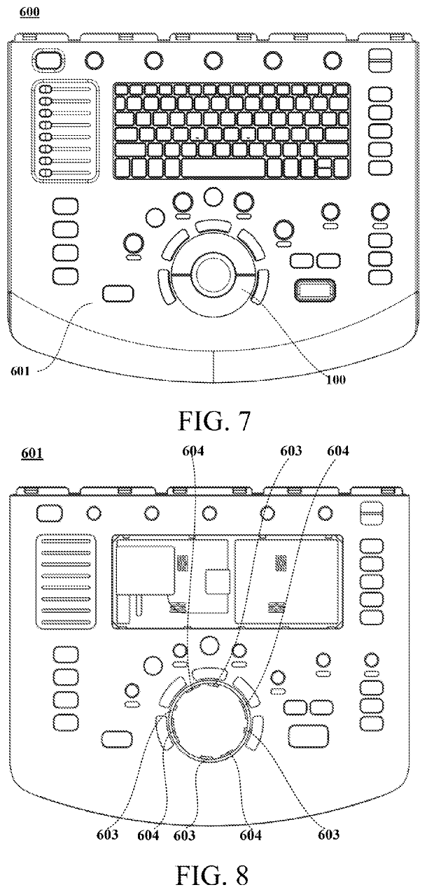

FIG. 6 is a schematic diagram of a medical input apparatus 600 according to an embodiment of the present application, and shows a situation before the trackball apparatus 100 is mounted in an operation panel 601. FIG. 7 is another schematic diagram of the medical input apparatus 600 according to an embodiment of the present application, and shows a situation after the trackball apparatus 100 is mounted in the operation panel 601.

As shown in FIG. 6, the operation panel 601 has a frame 602 for the trackball apparatus 100 to be mounted therein, and one or more position-limiting portions 603 matching the engaging portions of the trackball apparatus 100 are provided inside the frame 602. As shown in FIG. 6, corresponding to a cover plate 101, the frame 602 has a cylindrical shape, for example. However, the present application is not limited thereto. For example, the frame 602 may have a square shape, or any shape as long as the shape can match the shape of the cover plate 101.

In this way, the trackball apparatus can be easily detached and mounted, thereby facilitating maintenance and product replacement, and facilitating cleaning and maintenance of the inside of the trackball apparatus.

In some embodiments, as shown in FIG. 6, the position-limiting portion 603 includes a first protrusion portion 6031 extending in an axial direction. When the trackball apparatus 100 is mounted in the frame 602, the first protrusion portion 6031 is inserted into a recess 403 (see FIG. 4) of an engaging portion 1013 of the trackball apparatus 100.

In this way, engagement reliability can be further improved, and the trackball apparatus can be more easily mounted or detached.

In some embodiments, the position-limiting portion 603 further includes a second protrusion portion 6032 extending in a circumferential direction. When the trackball apparatus 100 is mounted in the frame 602, the second protrusion portion 6032 is inserted into a gap (see the gap S in FIG. 4) between the second surface 1012 of the cover plate 101 of the trackball apparatus 100 and the recess 403 of the engaging portion 1013 of the trackball apparatus 100.

In this way, the gap S between the recess 403 and the second surface 1012 accommodates the second protrusion portion 6032, and the second protrusion portion 6032 can block movement of the engaging portion 1013 in the axial direction, and can limit the position of the trackball apparatus 100 in the axial direction. Therefore, the trackball apparatus can be restricted from falling off from the frame of the operation panel in the axial direction, so that engagement reliability can be further improved, and the trackball apparatus can be more easily mounted or detached.

It should be noted that FIG. 6 and FIG. 7 merely schematically illustrate the embodiments of the present application, but the present application is not limited thereto. For example, each component may be adjusted appropriately, and some other components may be incorporated or some components may be eliminated. Those skilled in the art could make appropriate variations according to the above content, rather than being limited by the disclosure of FIG. 6 and FIG. 7.

The medical input apparatus is schematically described above, and the operation panel will be further described below.

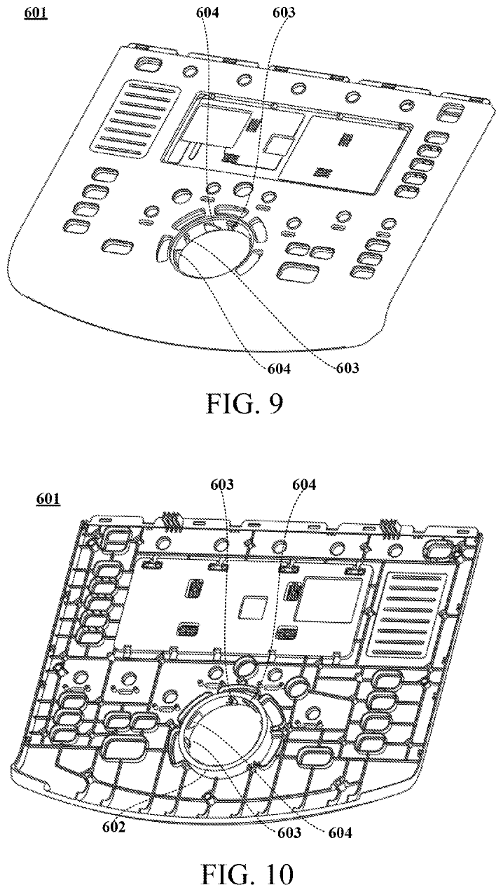

FIG. 8 is a schematic diagram of an operation panel according to an embodiment of the present application, and shows the operation panel 601 viewed from above. FIG. 9 is another schematic diagram of the operation panel according to an embodiment of the present application, and shows the operation panel 601 viewed from an upper side. FIG. 10 is another schematic diagram of the operation panel according to an embodiment of the present application, and shows the operation panel 601 viewed from below.

In some embodiments, as shown in FIG. 8 to FIG. 10, a plurality of position-limiting portions 603 may be symmetrically provided in a circumferential direction. When the trackball apparatus 100 is mounted in the frame 602, the position-limiting portions 603 are engaged with the engaging portions 1013 of the trackball apparatus 100. For example, the position-limiting portion 603 is inserted into the recess 403 of the engaging portion 1013.

In this way, engagement reliability can be further improved, and the trackball apparatus can be more easily mounted or detached.

In some embodiments, as shown in FIG. 8 to FIG. 10, a guiding member 604 is further provided inside the frame 602. When the trackball apparatus 100 is mounted in the frame 602, the guiding member 604 contacts the cut-out portion 404 of the engaging portion 1013 to guide the engaging portion 1013, such that the position-limiting portion 603 can be easily inserted into the recess 403 of the engaging portion 1013.

In addition, when the trackball apparatus 100 needs to be detached, the trackball apparatus 100 only needs to be rotated in an opposite direction, so that the cut-out portion 404 can move along the guiding member 604. Therefore, the trackball apparatus 100 can be lifted in the axial direction, such that a user can easily remove the trackball apparatus 100 from the housing 602.

For example, as shown in FIG. 8 to FIG. 10, the guiding member 604 is a protrusion having a slope, and the position-limiting portion 603 is a columnar protrusion, but the present application is not limited thereto. As long as the guiding member 604 can guide the engaging portion 1013, and the position-limiting portion 603 and the engaging portion 1013 can match together to implement an engagement function, the specific shapes thereof are not limited in the embodiments of the present application.

In this way, guidance can be performed through the guiding member and the cut-out portion. Engagement reliability can be further improved, and the trackball apparatus can be more easily mounted or detached.

It should be noted that FIG. 8 to FIG. 10 merely schematically illustrate the embodiments of the present application, but the present application is not limited thereto. For example, each component may be adjusted appropriately, and some other components may be incorporated or some components may be eliminated. Those skilled in the art could make appropriate variations according to the above content, rather than being limited by the disclosure of FIG. 8 to FIG. 10.

The above embodiments merely provide illustrative description of the embodiments of the present application. However, the present application is not limited thereto, and appropriate variations may be made on the basis of the above embodiments. For example, each of the above embodiments may be used independently, or one or more of the above embodiments may be combined.

As can be seen from the above embodiments, the second surface of the cover plate is provided with the engaging portions, and the engaging portions are located on an outer side of the housing when the cover plate is mounted on the housing. Engaging members matching the engaging portions are provided inside the frame of the operation panel. In this way, the trackball apparatus can be easily detached and mounted, thereby facilitating maintenance and product replacement.

An embodiment of the present application further provides an ultrasonic imaging apparatus, including the medical input apparatus as described in the forgoing embodiments, and the same content as that of the forgoing embodiments is not repeated herein.

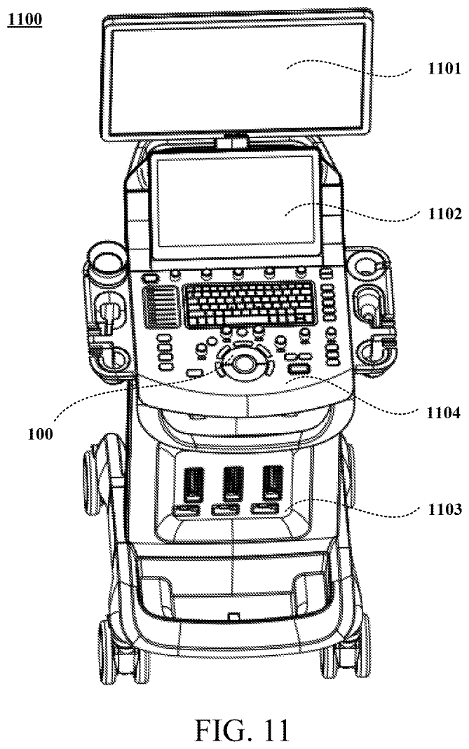

FIG. 11 is a schematic diagram of an ultrasonic imaging apparatus according to an embodiment of the present application. As shown in FIG. 11, an ultrasonic imaging apparatus 1100 may include a flat screen or display 1101, an independent touch screen 1102, and an operation panel 1104 usually mounted on a cart 1103.

For example, the flat screen or display 1101 displays one or more ultrasonic images obtained by the ultrasonic imaging apparatus 100. In addition, the flat screen or display 1101 may include a graphical user interface (GUI) used in combination with the operation panel 1104. The touch screen 1102 is used by a user or a clinician to configure or adjust settings of one or more ultrasound scanning apparatuses. The operation panel 1104 includes physical buttons and a trackball for interfacing with the GUI of the flat screen or display 1101. On the operation panel 1104, the trackball apparatus 100 according to the embodiment of a first aspect is configured.

It should be noted that FIG. 11 merely schematically illustrates the embodiment of the present application, but the present application is not limited thereto. For example, each component may be adjusted appropriately, and some other components may be incorporated or some components may be eliminated. Those skilled in the art could make appropriate variations according to the above disclosure, rather than being limited by the disclosure of FIG. 11.

The embodiments of the present application are described above with reference to specific implementations. However, it should be clear to those skilled in the art that such description is merely illustrative and is not intended to limit the scope of protection of the embodiments of the present application. Various variations and modifications could be made by those skilled in the art according to the spirit and principle of the embodiments of the present application, and these variations and modifications also fall within the scope of the embodiments of the present application.

Preferred implementations of the embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, because many modifications and changes could be easily conceived of by those skilled in the art, implementations of the embodiments of the present application are not limited to the illustrated and described precise structures and operations, but can cover all appropriate modifications and equivalents that fall within the scope of the implementations.

1. A trackball apparatus comprising:
a cover plate having a first surface and a second surface, wherein an opening portion passing through the first surface and the second surface is provided at the center of the cover plate, and one or more engaging portions are provided on the second surface;
a housing having an accommodating portion, wherein the cover plate contacts an end of the housing via the second surface such that the cover plate is mounted on the housing, and when the cover plate is mounted on the housing, the one or more engaging portions are located on an outer side of the housing; and
a ball accommodated in the accommodating portion in a freely rotatable state, wherein a part of the ball is exposed from the opening portion.

2. The trackball apparatus according to claim 1, wherein the first surface of the cover plate has a force receiving portion for applying a rotational force to the cover plate.

3. The trackball apparatus according to claim 1, wherein a detector is provided in the accommodating portion of the housing, and is used to detect input information generated by rotation of the ball.

4. The trackball apparatus according to claim 1, wherein the engaging portion has a plate-shaped portion extending from the second surface in an axial direction, and a recess extending in the axial direction is provided on a peripheral side of the plate-shaped portion.

5. The trackball apparatus according to claim 4, wherein a reinforcing portion extending in a radial direction is provided on a side of the plate-shaped portion close to the second surface, and a cut-out portion is provided on another peripheral side of the plate-shaped portion.

6. The trackball apparatus according to claim 5, wherein the recess does not contact the second surface, and the cut-out portion is located away from the second surface in the axial direction.

7. The trackball apparatus according to claim 1, wherein a mounting portion is further provided on the second surface of the cover plate, and the cover plate contacts an end of the housing via the mounting portion such that the cover plate is mounted on the housing.

8. The trackball apparatus according to claim 1, wherein the apparatus further comprises a fixing member having a circular opening, the diameter of the circular opening of the fixing member is less than the diameter of the ball, and the fixing member is detachably connected to the housing; and when the cover plate is mounted on the housing, the fixing member is located inside the opening portion, and the part of the ball is exposed from the circular opening of the fixing member.

9. A medical input apparatus comprising:
a trackball apparatus comprising:
a cover plate having a first surface and a second surface, wherein an opening portion passing through the first surface and the second surface is provided at the center of the cover plate, and one or more engaging portions are provided on the second surface;
a housing having an accommodating portion, wherein the cover plate contacts an end of the housing via the second surface such that the cover plate is mounted on the housing, and when the cover plate is mounted on the housing, the one or more engaging portions are located on an outer side of the housing; and
a ball accommodated in the accommodating portion in a freely rotatable state, wherein a part of the ball is exposed from the opening portion; and
an operation panel having a frame for the trackball apparatus to be mounted therein, wherein one or more position-limiting portions matching the engaging portions of the trackball apparatus are provided inside the frame, and when the trackball apparatus is mounted in the frame, the position-limiting portions are engaged with the engaging portions of the trackball apparatus.

10. The medical input apparatus according to claim 9, wherein the position-limiting portion comprises a first protrusion portion extending in an axial direction; and when the trackball apparatus is mounted in the frame, the first protrusion portion is inserted into the recess of the engaging portion of the trackball apparatus.

11. The medical input apparatus according to claim 10, wherein the position-limiting portion further comprises a second protrusion portion extending in a circumferential direction; and when the trackball apparatus is mounted in the frame, the second protrusion portion is inserted into a gap between the second surface of the cover plate of the trackball apparatus and the recess of the engaging portion of the trackball apparatus.

12. The medical input apparatus according to claim 10, wherein a guiding member is further provided inside the frame, and when the trackball apparatus is mounted in the frame, the guiding member contacts the cut-out portion of the engaging portion to guide the engaging portion.

13. The medical input apparatus according to claim 9, wherein the first surface of the cover plate has a force receiving portion for applying a rotational force to the cover plate.

14. The medical input apparatus according to claim 9, wherein a detector is provided in the accommodating portion of the housing, and is used to detect input information generated by rotation of the ball.

15. The medical input apparatus according to claim 9, wherein the engaging portion has a plate-shaped portion extending from the second surface in an axial direction, and a recess extending in the axial direction is provided on a peripheral side of the plate-shaped portion.

16. An ultrasonic imaging apparatus comprising:
a trackball apparatus comprising:
a cover plate having a first surface and a second surface, wherein an opening portion passing through the first surface and the second surface is provided at the center of the cover plate, and one or more engaging portions are provided on the second surface;
a housing having an accommodating portion, wherein the cover plate contacts an end of the housing via the second surface such that the cover plate is mounted on the housing, and when the cover plate is mounted on the housing, the one or more engaging portions are located on an outer side of the housing; and
a ball accommodated in the accommodating portion in a freely rotatable state, wherein a part of the ball is exposed from the opening portion; and
an operation panel having a frame for the trackball apparatus to be mounted therein, wherein one or more position-limiting portions matching the engaging portions of the trackball apparatus are provided inside the frame, and when the trackball apparatus is mounted in the frame, the position-limiting portions are engaged with the engaging portions of the trackball apparatus.

17. The ultrasonic imaging apparatus according to claim 16, wherein the position-limiting portion comprises a first protrusion portion extending in an axial direction; and when the trackball apparatus is mounted in the frame, the first protrusion portion is inserted into the recess of the engaging portion of the trackball apparatus.

18. The ultrasonic imaging apparatus according to claim 16, wherein the first surface of the cover plate has a force receiving portion for applying a rotational force to the cover plate.

19. The ultrasonic imaging apparatus according to claim 16, wherein a detector is provided in the accommodating portion of the housing, and is used to detect input information generated by rotation of the ball.

20. The ultrasonic imaging apparatus according to claim 16, wherein the engaging portion has a plate-shaped portion extending from the second surface in an axial direction, and a recess extending in the axial direction is provided on a peripheral side of the plate-shaped portion.
